(19) United States
(12) Patent Application Publication
(10) Pub. No.: US 2006/0272640 A1
(43) Pub. Date: Dec. 7, 2006

(54) Anatomical nasal inhaler

(76) Inventor: Salvador Tirado Abullon, Madrid (ES)

Correspondence Address: Salvador Tirado Abullon, Victor De La Serna 3 5 Parking, Madrid, 28016, ES

(21) Appl. No.: 10/551,773

(22) PCT Filed: Apr. 26, 2005
(86) PCT No.: PCT/ES05/00220

§ 371 Date: Oct. 3, 2005

(30) Foreign Application Priority Data
Apr. 26, 2004 (ES) P200400992

(51) Int. Cl. A61M 15/08; A61G 10/00

(52) U.S. Cl. 128/203.22; 128/206.11

(57) ABSTRACT

This inhaler (1) is composed of two parallel cylindrical tubes (1.1), a linking bridge (2) between said cylinders, and two threaded terminals (10, 11).

The inhaler is provided with three peripheral rings, one external one (5), one intermediate one (6) (located within each cylindrical tube) and one internal one (7), located in the terminal (10, 11), the section of which has a semicircular shape with a diameter of 2 mm. The planes of the external openings of the cylindrical tubes (1.1), form an angle of 130°.

The internal opening (4) of each cylinder is provided with a threaded terminal with two standard lengths of 5 mm and 9 mm, which a variable in other versions. The average exterior diameter of the terminals is of 10 mm, although it can vary between 8 y 14 mm, at intervals of 2 mm.

### OBJECT OF THE INVENTION

This invention relates to a new type of anatomical inhaler, which when introduced into the nasal cavities, facilitates breathing for people who for different reasons have said cavities totally or partially obstructed.

### BACKGROUND OF THE INVENTION

We have all had occasion, at some point in our lives, to experiment the discomfort of a stuffed nose. Doubtless there exists an instinctive desire to breathe through one's nose. Attempting, when one's nose is blocked, to overcome the resistance put up by the nasal passages against the movement of air is associated with a feeling of general discomfort and tiredness. The normal functioning of the entire respiratory system depends on adequate nasal permeability.

In a very general way, we could define nasal obstruction as a situation in which a sufficient quantity of air does not reach the lungs upon breathing exclusively through one's nose.

In new-borns and lactating infants, a nasal blockage, even if temporary, can create a considerable concern due to its severity, since infants are nasal breathers that do not know how to breathe through their mouths, and in addition they need to have an unblocked nose in order to be fed correctly. That is, in order to suckle the mother's breast or a bottle, a sufficient passage of air is required by way of the nose. Bigger children are better able to tolerate nasal obstruction, although if it is chronic it can cause malformation in their mouths and face. Normally this nasal obstruction is brought about by large or inflamed adenoid vegetation due to a nasal infection, to foreign bodies that children often put in their noses, but it can also be provoked by birth defects such as the lack of perforation of the posterior nasal orifice or choanal atresia, tumours, traumatism or many other causes. On many occasions large and inflamed adenoid pharyngeal polypus are accompanied by inflammation of the mucous of the middle ear, provoking the accumulation of serosity and mucosity in the tympanum, which is known by the name of otitis secretory or mucous-secretory otitis. Frequently, such conditions are not painful, although they always bring about a more or less severe degree of hypoacusia or hearing loss. Such cases, very often, must be treated surgically, undertaking the removal, under general anaesthesia, of the pharyngeal polypus together with a small incision made, under the control of the microscope, in the tympanum membrane, in order to suck up the mucous from the middle ear and insert a ventilation tube. Especially in children, any obstruction of the nasal cavities must be studied and treated correctly in order to avoid serious complications, in very small children, and mouth and face deformation, in older children.

In adults, nasal obstruction can be due, also, to many causes. Infections caused by a virus or bacteria, especially sinusitis, both benign and malign tumours, traumatism, nose malformations, metabolic disorders, allergies, etc. should all be mentioned.

In a practical way, it can be summed up that a nasal obstruction is either due to a disorder in the functioning of erectile structures, located within the nasal cavities and called nasal concha, within which should be considered inflammations of nasal mucous provoked by allergies, medication, irritating substances, infections, etc., or to a disorder of the anatomical structures of the nose, both within (nasal cavity and nasal wall), and without (nasal cone).

The treatment of nasal obstructions provoked by a functional disorder will be medical and vary according to the cause that has provoked it; likewise, when nasal obstruction is provoked by an anatomical disorder of the nasal structures, the treatment will be surgical in the majority of cases. The type of surgical intervention will obviously also depend on the cause that provokes it.

Although it is true that there is a great number of nasal obstructions that do not require any treatment since they heal spontaneously, it should be emphasised that a doctor should be consulted regarding any persistent nasal obstruction, since it can be either a symptom of a banal cause, or the manifestation of a severe illness that requires early medical or surgical treatment.

For cases of temporary nasal obstruction where medical treatment is not immediately required, there are nasal strips that consist of a kind of gauze made up of two plastic filaments which, upon being folded over the nasal bone, create resistance by way of which the nostril wings are opened.

According to studies carried out at the Department of Otorhinolaryngology of the University of Ulleval, in Oslo (Norway), Dr. Djupesland has demonstrated that these nasal strips can “provide benefits to persons who suffer from slight snoring and objective nasal obstruction, caused by seasonal allergies or rhinitis, although those who suffer severe apneas or have a very large nose will probably not notice the difference if they use the dilator”. Likewise, pregnant women in the final months of gestation can also benefit from dilators.

The Norwegian specialist explains that his double blind study, with placebo, included 20 patients with objective nasal obstruction of an average of 35% throughout the entire night.

The subjective evaluation of the usefulness of the nasal strips implied a significant improvement in sleep quality, reduction of mouth dryness and greater sensation of a clear nose. The objective measurements taken by way of acoustic revealed an increase in the size of the nostril passages that persisted throughout the night.

Another noteworthy result of the study was that the beneficial action of the nasal strips increased when the patients observed took up a lateral sleeping posture, in contrast to a supine one.

Other studies have questioned the usefulness of nasal strips, such as those carried out by the University of Buffalo (U.S.A.), coordinated by Dr. Frank Cerny. In this study it is demonstrated that the use of strips in athletes, improved air capture is not produced, nor, hence, is their performance improved.

In this sense, Dr. Djupesland has affirmed that the usefulness of dilators is just one factor among many others that are combined in the remission of snoring, such as a careful diet, not being overweight, tobacco or alcohol, especially before going to sleep. All in all, the otorhino-laryngologist has affirmed that this method is palliative for relief of slight snoring, and should not be considered to be a solution for more severe problems such as apnea.

With the objective of solving the problems described above, a new type of mechanism has been developed that substantially improves breathing in cases in which the affected person should have an anatomical, morphological and physiological condition of the nose itself, smoking habits, winter pathologies, etc. that may bring about a ventilation insufficiency of the nasal cavities. This implicates compensating the respiratory function by breathing through the mouth, motivating disorders such as mouth dryness, night snoring, sleep disorders, etc.

On the other hand, limitations in physical exercise and sporting activity, in general, can be impairment caused by respiratory insufficiency through the nasal passages.

### DESCRIPTION OF THE INVENTION

The inhaler of the invention is made up of two empty cylinders, with a completely smooth interior surface, generally parallel and joined by a linking bridge, and two terminals, one for each cylinder.

At the end of each cylinder corresponding to the part that enters more deeply into the nasal passage, the cylinder is threaded on the exterior; the length of the threaded zone is less than 5 mm, preferably of 3 mm.

On each aforementioned end area of each cylinder, a cylindrical terminal is screwed on and is threaded on its interior, which may have different lengths, although there are two standard lengths: a shorter length of 5 mm, and a longer one of 9 mm, although they can be made with lengths varying by intervals of 2 mm.

The mounted assembly formed by each cylinder and its corresponding threaded terminal presents three peripheral rings, one external and the other intermediate, located on each cylinder, and one internal (located at the threaded terminal, on the part of the inhaler that it inserted more deeply into the nasal cavity) with a semicircular section of a diameter of about 2 mm.

The functions of the rings are to facilitate the introduction of the inhaler in the nose and maintain a certain separation between the inner wall of the nasal cavities and the outer wall of the cylindrical tubes, as well as to adjust itself to the nasal cavities in a comfortable and secure way.

The ends of the cylinders that coincide with the external and internal rings are the apertures through wich air enters and exits during the phases of inhaling and exhaling.

The exterior average diameter of each cylinder and its corresponding terminal is of 10 mm, although it can vary between 8 and 14 mm, on a scale with intervals of 2 mm.

The size of each terminal is chosen depending on each type of nose in order to facilitate its anatomical adaptability, such as in cases of small noses, prominent ones, etc.

The external planes of aperture of the two cylinders form an angle of 130°, as can be observed in FIG. 1B (angle α). In consequence, the contour of each external aperture is elliptical.

The linking bridge between the two cylinders, in the shape of an arch or semicircle, can include a thickening in its centre-front area, with a flexible axis in its interior, for a more personalised adjustment. Said bridge has several functions:
it acts as a limiting stopper of the depth of penetration of the cylinders into the nasal cavities.
it also servers as a handle that facilitates both applying and extracting the inhaler.

The appropriate material for producing this mechanism is preferably rigid or semi-rigid plastic, variable in its components.

The inhaler of the invention can feature threaded terminals with a self-blocking system that prevents them from accidentally unscrewing; by way of application with the fingers of a slight pressure on the periphery of the terminals, they are unblocked, thus allowing them to be unscrewed easily.

The objective of this blocking is to provide total security to the user, especially if it is a child, regarding the possibility that the terminal should be loosened within the nose; the possibility of unblocking the screwed-in terminal allows it to be substituted when the use of another one with a different length is required.

### DESCRIPTION OF THE DRAWINGS

In order to complement the description of this invention, and with the objective of improving the understanding of its characteristics, it is accompanied by a series of figures in which, with a purely illustrative and non-limiting purpose, the following pictures are represented, the main components of which are the following.
(1) Inhaler
   (1.1) Cylindrical tube
(2) Bridge
(3) External opening
(4) Internal opening
(5) External ring
(6) Intermediate ring
(7) Internal ring
(8) Threaded area of the cylinder (1.1)
(9) Angle
(10) Short terminal
(11) Long terminal
(12) Male stopper
(13) Female stopper

FIG. 1A is a front view of the inhaler (1) (once it has been situated inside the nose).

FIG. 1B, is an elevated view of the inhaler (1), with the short terminals (10) unscrewed.

FIG. 1C is an elevated view of the short terminals (10) of the inhaler (1).

FIG. 1D is the view from the A-A symmetry plane of a section of the inhaler (1) of FIG. 1B.

FIG. 1E is an elevated view of the inhaler (1) with the short terminals (10) screwed in.

FIG. 1F is a lateral profile view of the inhaler (1), with the short terminals (10) screwed in.

FIG. 2A is a front view of the inhaler (1) (once it has been situated inside the nose).

FIG. 2B is an elevated view of the inhaler (1), with the long terminals (11) unscrewed.

FIG. 2C is an elevated view of the long terminals (11) of the inhaler (1).

FIG. 2D is the view from the A-A symmetry plane of a section of the inhaler (1) of FIG. 2B.

FIG. 2E is the elevated view of the inhaler (1) with the long terminals (11) screwed in.

FIG. 2F is a lateral profile view of the inhaler (1) with the long terminals (11) screwed in.

FIG. 3A is an elevated view of a type of inhaler (1) characterised in that it is provided with a self-blocking system made up of female stoppers (13) and male stoppers (12).

FIG. 3B is a section by the A-A plane of FIG. 3A, where the male stoppers (12) are shown.

FIG. 3C is an elevated view of the threaded terminal, in which the female stopper (13) is detailed.

FIG. 3D is a section from the B-B plane of FIG. 3C, in which the female stopper (13) is detailed.

FIG. 4 is the view from a perspective of the inhaler drawn in an ideal position for introduction into the nasal cavities.

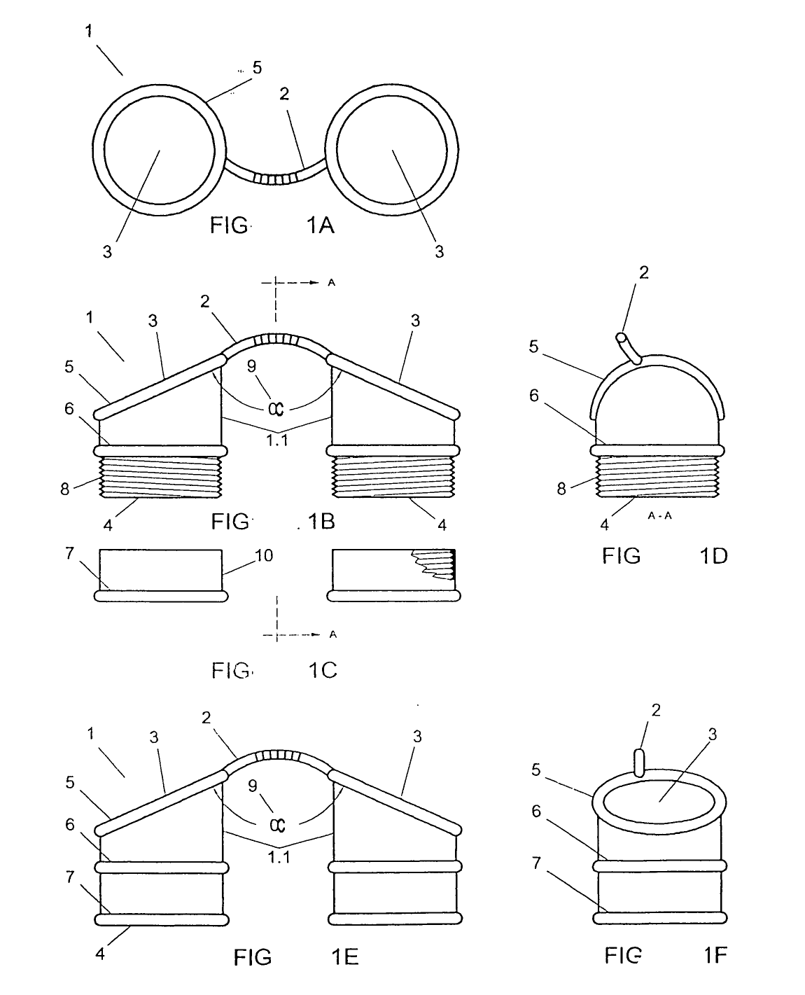

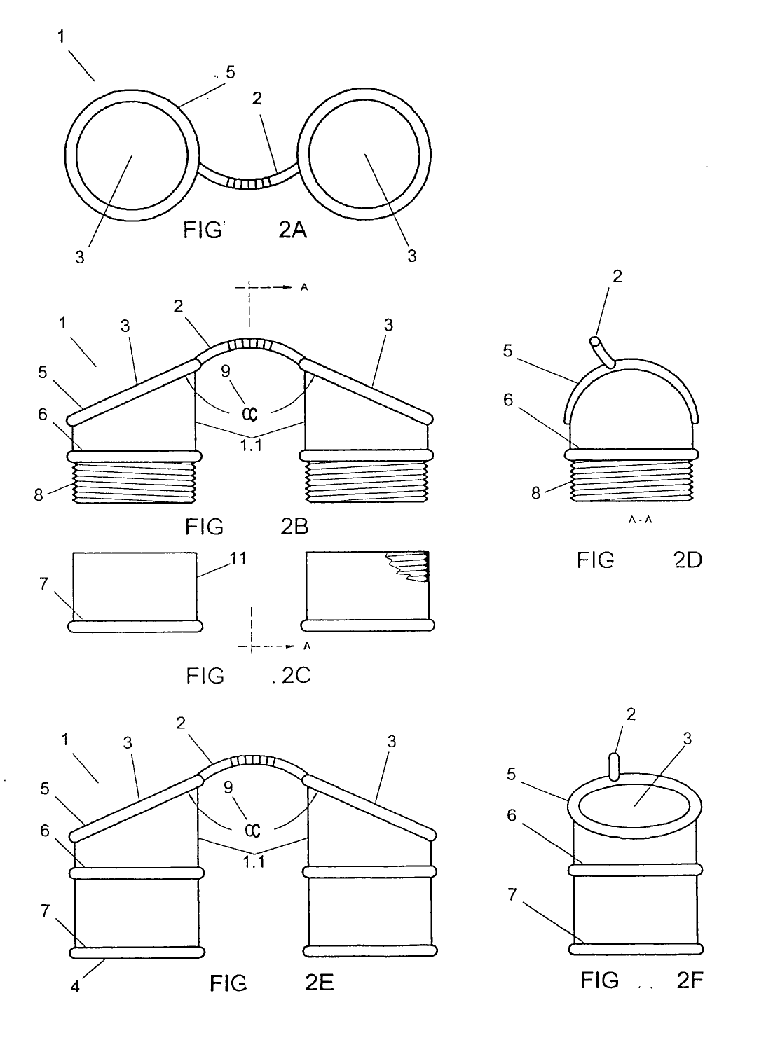

### PREFERRED EMBODIMENT OF THE INVENTION

Among the different types of anatomical inhalers that can be produced, taking this invention as a basis, the preferred embodiment is the one described below.

This inhaler (1) is characterised by being composed of two cylindrical tubes (1.1), one for each nasal orifice, joined by a linking bridge (2), and two threaded terminals (10, 11). At the end area (8) of each cylinder corresponding to the part that enters more deeply into the nasal cavity, the cylinder is threaded on its exterior. The length of said threaded end area (8) is less than 5 mm, and is preferably 3 mm.

Both cylindrical tubes (1.1) are joined at their anterior or front area by a linking bridge (2) in the shape of an arch or semicircle. This bridge (2) serves as an external stopper on the wall of the nose to prevent the internal progression of the cylinders. At the same time it serves as a handle or holder, for both its application and its extraction.

This bridge (2) or linking component is made up of a strip with a circular section with an equal diameter to that of the external ring (5) of the cylindrical tube (1.1); it may also have a widening at its front-central area, with a flexible axis in its interior for a more personalised fit.

The cylindrical tube (1.1) of each nasal orifice, open at its two ends, is provided with an external opening (3) and with an internal opening (4).

The planes of the external opening (3) form a 130° angle (9).

The external opening (3) of the cylindrical tube (1.1) is provided with an external peripheral ring (5) and, at the area alongside the beginning of the threaded area (8), each cylindrical tube (1.1) is also provided with an intermediate (6), which is also peripheral and constitutes the stopper of the threaded terminal (10, 11). Said threaded terminal, in turn, is provided on its end corresponding to the internal opening (4) of each cylinder, with an internal peripheral ring (7), in order for the inhaler (1) to be adjust in an easy, comfortable and safe way.

All of these rings have a semicircular section with a diameter of about 2 mm.

There are two types of terminals. Short terminals (10) with a length of 5 mm, and long terminals (11) with a length of 9 mm. Both terminals have the same exterior diameter, of 10 mm, in the standard version.

Due to the rounded form that the external (5) and internal (7) rings have, it is very easy to introduce the inhaler into ones nasal cavities in a comfortable and safe way.

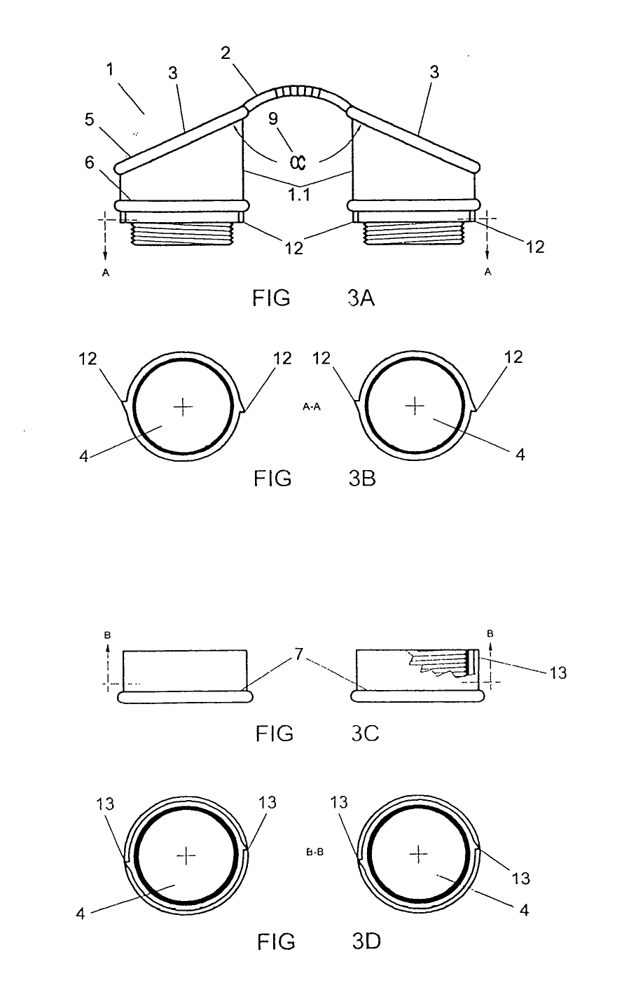

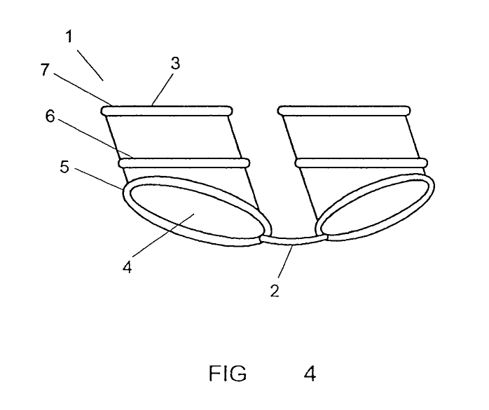

In another preferred embodiment, the threaded terminals (10, 11) are provided with a self-blocking system of a conventional kind that prevents them from accidentally becoming unscrewed; this system can be constituted, among the many existing alternatives for blocking a pair of threaded parts in a reversible way, as indicated in FIGS. 3A to 3D, by way of shoulders or male stoppers (12) arranged on the periphery of the threaded area (8) of each cylindrical tube (1.1), which, when the terminals (10, 11) are completely threaded, are introduced into notches or female stoppers (13) located at the internal peripheral area of said terminals (10, 11). By applying slight pressure with ones fingers on the periphery of the terminals, they unblock allowing them to be easily unscrewed.

The object of this blockage is to provide total security to the user, especially if it is a child, regarding the possibility that the terminal should be loosened inside their nose; the possibility of unblocking the threaded terminal allows it to be replaced when one of a different length needs to be used.

For all these reasons, the anatomical nasal inhaler can be applied with particular, advantageous features with respect to the previously known solutions.

Having sufficiently described the nature of the present invention, as well as a practical embodiment of the same, it only needs to be added that modifications may be introduced in its shape, size, materials and production procedure, as long as they do not substantially affect the characteristics claimed below.

1.- Anatomical nasal inhaler of the type that are partially introduced into the nasal cavities in order to facilitate breathing, characterised in that is made up of:
two cylindrical tubes (1.1) generally parallel, each with and end area (8) that is threaded on its exterior at a length less than 5 mm
a linking bridge (2) between said cylinders, and
two cylindrical terminals (10, 11) threaded in their interior.

2.- Anatomical nasal inhaler according to claim 1, characterised in that each cylindrical tube (1.1) is provided with an internal opening (4) with a circular contour and with an external opening (4) with an elliptical contour.

3.- Anatomical nasal inhaler according to claim 1, characterised in that said threaded terminals (10, 11) have a variable height depending on the inhaler model.

4.- Anatomical nasal inhaler according to claim 1, characterised in that each cylindrical tube (1.1) is provided with two peripheral rings: one is external one (5) located at the external opening (3) of the cylindrical tube (1.1), and one intermediate one (6), located in the area of said cylindrical tube (1.1) alongside the start of the threaded area; and in that each terminal (10, 11) is provided at the end corresponding to the internal opening (4) of each cylindrical tube (1.1) of an internal peripheral ring (7) and at its opposite end, functions as a stopper against the intermediate ring (6).

5.- Anatomical nasal inhaler according to claim 4, characterised in that the section of said peripheral rings (5, 6, 7) is semicircular with a diameter of 2 mm.

6.- Anatomical nasal inhaler according to claim 2, characterised in that the planes of the external openings (3) with elliptical contour of the cylindrical tubes (1.1) that make up the anatomical nasal inhaler form an angle of 130°.

7.- Anatomical nasal inhaler according to claim 1, characterised in that the interior wall of each cylindrical tube (1.1) is totally smooth.

8.- Anatomical nasal inhaler according to claim 1, characterised in that the linking bridge (2) of the two cylinders (1.1), in the standard version of the inhaler, is made up of a strip with a circular section with a diameter equal to that of said external ring (5).

9.- Anatomical nasal inhaler according to claim 1, characterised in that the linking bridge (2) of the two cylinders (1.1) in another version of the inhaler, is provided with a widening at its centre-front area, with a flexible axis in its interior.

10.- Anatomical nasal inhaler according to claim 1, characterised in addition in that in the periphery of the threaded area (8) of each cylindrical tube (1.1) male shoulders or stoppers (12) are situated, which, when the terminals (10, 11) are completely screwed in, are introduced into notches or female stoppers (13) located at the internal periphery of said terminals (10, 11) in order to prevent said terminals (10, 11) from being accidentally unscrewed.
